(19) United States
(12) Patent Application Publication
(10) Pub. No.: US 2010/0135945 A1
(43) Pub. Date: Jun. 3, 2010

(54) GYMNEMA-CONTAINING LIP BALM COMPOSITIONS AND ASSOCIATED METHODS

(75) Inventors: Kristin Kelly Murdock, Provo, UT (US); Angela D. Murdock, Highland, UT (US)

Correspondence Address: STOEL RIVES LLP - SLC, 201 SOUTH MAIN STREET, SUITE 1100, ONE UTAH CENTER, SALT LAKE CITY, UT, 84111, US

(73) Assignee: KREATIONS BY KRISTIN, LLC, Springville, UT (US)

(21) Appl. No.: 12/628,896

(22) Filed: Dec. 1, 2009

Related U.S. Application Data
(60) Provisional application No. 61/119,177, filed on Dec. 2, 2008.

(51) Int. Cl. A61K 8/18; A61K 36/00; A61Q 1/06; A61K 36/752; A61K 36/534; A61K 36/53; A61K 36/899; A61K 36/736; A61K 36/886; A61K 36/28; A61K 36/47

(52) U.S. Cl. 424/64; 424/725; 424/736; 424/747; 424/745; 424/750; 424/735; 424/744; 424/764; 424/775; 424/731

(57) ABSTRACT

Extracts of members of the plant family Asclepiadaceae can alter or diminish a person's ability to taste some sugars. As such, the extracts can be used help a person reduce their caloric intake and thereby reduce their body weight or body fat percentage. An extract of the plant Gymnema sylvestre can be used as an ingredient in a lip balm. The Gymnema-containing lip balm can be applied to a person's lips before they eat a sugar-containing food item. The lip balm can also contain other weight-loss promoting substances such as jasmine, grapefruit, vanilla, lemon, peppermint, oregano, ylang ylang, and combinations of the preceding substances.

### CROSS-REFERENCE TO RELATED APPLICATIONS

The present application is a non-provisional of U.S. Provisional Application 61/119,177, filed Dec. 2, 2008, entitled GYMNEMA-CONTAINING LIP BALM COMPOSITIONS AND ASSOCIATED METHODS.

### TECHNICAL FIELD

The present disclosure relates generally to the fields of cosmetics, nutritional supplements, and weight loss aids. More specifically, the present disclosure relates to compositions comprising Gymnema sylvestre extract for use in weight loss.

### DETAILED DESCRIPTION OF PREFERRED EMBODIMENTS

It will be readily understood that the components of the embodiments as generally described herein could be arranged and designed in a wide variety of different configurations. Thus, the following more detailed description of various embodiments is not intended to limit the scope of the disclosure, as claimed, but is merely representative of various embodiments.

In one embodiment, a composition comprises a medicament having an extract of the plant family Asclepiadaceae. In another embodiment, a composition comprises a weight-loss aid comprising an extract of the plant family Asclepiadaceae. In another embodiment, a composition comprises a formulation that alters or distorts the flavor of food. In another embodiment, a composition comprises a formulation that alters a person's perception of the tastes of some sugars.

In another embodiment, a composition comprising an extract of the family Asclepiadaceae can be formulated as a cosmetic. In another embodiment, a composition comprising an extract of the plant family Asclepiadaceae is formulated such that it can be applied to a person's lips. In one embodiment, a composition comprising an extract of the family Asclepiadaceae can be formulated as a lip balm. In one embodiment, a composition comprising an extract of the family Asclepiadaceae can be formulated as a lip gloss. In one embodiment, a composition comprising an extract of the family Asclepiadaceae can be formulated as a lipstick. In one embodiment, a composition comprising an extract of the family Asclepiadaceae can be formulated as a lip salve.

The present disclosure also provides a method for formulating a medicament comprising an extract of the family Asclepiadaceae. Another embodiment comprises a method for formulating a composition for weight-loss, wherein the composition comprises an extract of the family Asclepiadaceae. Another embodiment comprises a method for formulating a composition for altering a person's perception of taste, wherein the composition comprises an extract of the family Asclepiadaceae. Another embodiment comprises a method for formulating a composition that alters a person's perception of the taste of some sugars, wherein the composition comprises an extract of the family Asclepiadaceae.

The present disclosure also provides a method for reducing a person's weight via employing a composition comprising an extract of the family Asclepiadaceae. In one embodiment, a method for reducing a person's caloric intake comprises employing a composition comprising an extract of the family Asclepiadaceae. In one embodiment, a method for altering a person's perception of taste comprises employing a composition comprising an extract of the family Asclepiadaceae. In one embodiment, a method for altering a person's perception of the taste of some sugars comprises employing a composition comprising an extract of the family Asclepiadaceae. In one embodiment, a method for reducing person's intake of sugar comprises employing a composition comprising an extract of the family Asclepiadaceae.

In addition to an extract of the family Asclepiadaceae, compositions and formulations disclosed herein may comprise one or more additional substances known in the art for reducing the appetite, promoting weight loss, promoting additional caloric expenditures, or increasing thermogenesis. These substances reducing the appetite, promoting weight loss, promoting additional caloric expenditures, or increasing thermogenesis may include jasmine, grapefruit, vanilla, lemon, peppermint, oregano, ylang ylang, caffeine, green tea extract, theobromine, ginseng root and amino acids. Caffeine is known to promote weight loss by increasing thermogenesis and energy. Theobromine is found in products of the cocoa tree. It is found in chocolate products and used in medicine as a diuretic, vasodilator, and myocardial stimulant. It affects humans similarly to caffeine, but on a much smaller scale. Green tea has long been known to have an effect on body weight and energy expenditure. Epigallocatechin gallate (abbreviated herein as EGCG) is a naturally-occurring substance found chiefly in green tea and green tea extracts. EGCG is ideal as a weight loss agent because of its lack of toxicity or apparent side effects. EGCG and related catechins occur naturally in several types of plants, including tea, and thus have a long history of safety in that form. Ginseng root has been shown to have therapeutic value in increasing energy, reducing stress and improving memory. Amino acids such as theonine and arginine have also been found to promote weight loss. Theonine is known to have calming effects and promote a feeling of satiety. Arginine inhibits the absorption of dietary fat and is also known to combat fatigue.

Additional ingredients may be added to various compositions of the present disclosure, including, but not limited to anti-oxidants, vitamins, minerals and other trace elements. Additionally, artificial sweeteners, sugar alcohols and glycerol, organic acids, stabilizers and gums may also be added to various compositions of the present disclosure. A variety of supplements may also be added to the compositions. The formulations disclosed herein maybe used in connection with any active ingredients that suppress the appetite or promote weight and fat loss. Formulations of the present disclosure may also include natural and artificial flavors including, mint, chocolate, spice, salt, vanilla, citrus, fruit, herbs, coffee and other food flavors.

Compositions and formulations disclosed therein may comprise one or more additional substances such as, an extract of another plant that belongs to the family Asclepiadaceae, essential oil, flavor oil, perfume, suncreen, sunblock, colorant, and other auxiliaries and/or additives which are common for cosmetic formulations.

Essential oils and flavor oils may include tea tree, rosemary, sage, jasmine, peppermint, spearmint, anise, vanilla, cinnamon, camphor, lavender, ginger, grape, lemon, orange, grapefruit; almond, olive, castor, avocado, tamanu, emu, apricot kernel oregano, ylang ylang, or any combination of these oils. Flavor oils may be used at range of from about 0.01% to about 10%.

In one embodiment of the present disclosure the auxiliaries and/or additives which are common for cosmetic purposes are selected from the group consisting of oily bodies, surfactants, emulsifiers, fats, waxes, pearlescent waxes, bodying agents, thickeners, superfatting agents, stabilizers, polymers, silicone compounds, lecithins, phospholipids, biogenic active ingredients, deodorants, antimicrobial agents, hydrotropes, solubilizers, preservatives, perfume oils and dyes. Sorbitan oleate is an emulsifier that may be used in accordance with the present disclosure.

Oils, fats, or waxes may include almond oil, olive oil, castor oil, avocado oil, tamanu oil, emu oil, apricot kernel oil, shea oil, macadamia nut oil, candelilla wax, carnauba wax, beeswax, avocado butter, mango butter, cocoa butter, shea butter, or any combination of these or other substances known in the art. The various “butter” substances disclosed herein may each be described as being a “fat”. Fats and waxes can be described as being room temperature solids. Oils can be described as been room temperature liquids.

Substances for coloring compositions disclosed herein may comprise mineral pigments or organic dyes. The pigments may be dispersed in oil or glycerin. Coloring substances may include iron oxide, mica powder, D&C colors, bismuth oxychloride, ferric ammonium ferrocyanide, titanium dioxide, or a combination of these or any other suitable substances known in the art.

An extract of the family Asclepiadaceae may comprise an extract of Gymnema sylvestre. The herb Gymnema sylvestre and other members of the plant family Asclepiadaceae are known to comprise a number of compounds that can elicit various physiological responses in mammals, such as a reduction or elimination of a person's ability to taste some sugars. A Gymnema extract may alter, diminish, or eliminate a person's ability to taste some sweet foods by being contacted with the person's lips, mouth, and/or tongue. Additionally, some of the active ingredients in the Gymnema extract may be absorbed through a persons skin, for example, their lips, and elicit physiological responses that include altering a person's ability to taste some sugars.

The plant Gymnema sylvestre contains two resins (one of them is soluble in alcohol), gymnemic acids, saponins, stigmasterol, quercetol, and the amino acid derivatives betaine, choline and trimethylamine. An extract of the plant Gymnema sylvestre contains gymnemic acids, flavonoids and phenolic acids. Phenolic acids are acids that have a hydroxybenzene moiety in their structure, e.g. p-hydroxybenzoic acid, protocatechuic acid, vanillic acid, gallic acid, caffeic acid, p-coumaric acid, ferulic acid.

The concentration of the extract of the plant in the compositions for cosmetic or for medical applications can vary in wide ranges. In various embodiments of the present disclosure the concentration of Gymnema sylvestre extract can be between about 0.01% and about 50% by weight. In other embodiments, the concentration of gymnemic acid can be between about 0.01% and about 50% by weight.

One method of producing the extract of a plant that belongs to the family Asclepiadaceae (such as Gymnema sylvestre) according to the present disclosure comprises: extracting the plant or parts of the plant with a solvent selected from the group consisting of water, an alcohol with 1 to 6 carbon atoms (such as methanol), a 1,2-alkanediol (1,2-butanediol or 1,2-pentanediol) and mixtures thereof to obtain a mixture comprising the solvent and the extract; and removing the solvent from the mixture to obtain the extract.

Also, Gymnema sylvestre extracts may be obtained from a commercial supplier, such as Indo World Trading Corporation, which supplies a standardized Gymnema dry extract (25% in gymnemic acid, saponins, triterpens). In addition, the extract contains phenolic acids and flavonoids.

Additionally, a standardized dry extract can be obtained by extracting the leaves of the plant Gymnema sylvestre with water, or with alcohols or with mixtures of water and alcohols. The extract can be standardized so that its content in gymnemic acids is about 25% by weight. This extract may further contain derivatives of quercetol and acids derived from p-coumarine and phenol and vanillic acids and sinapic acids.

Substances disclosed herein can be combined in various Gymnema-containing compositions, such as a Gymnema-containing lip balm. Gymnema-containing lip balms may comprise Gymnema extract as disclosed herein or individual components of a Gymnema extract, such as gymnemic acid. Individual Gymnema extract components may be obtained via purification of a Gymnema extract, by sythesizing the component, or by some other process.

Gymnema-containing lip balms or lip sticks may comprise a variety of formulations. For example, in one embodiment, a Gymnema-containing lip balm comprises jasmine. In another embodiment, a Gymnema-containing lip balm comprises grapefruit. In another embodiment, a Gymnema-containing lip balm comprises vanilla. In another embodiment, a Gymnema-containing lip balm comprises lemon. In another embodiment, a Gymnema-containing lip balm comprises peppermint. In another embodiment, a Gymnema-containing lip balm comprises oregano. In another embodiment, a Gymnema-containing lip balm comprises ylang ylang. In another embodiment, a Gymnema-containing lip balm comprises caranuba wax. In another embodiment, a Gymnema-containing lip balm comprises candelilla wax. In another embodiment, a Gymnema-containing lip balm comprises beeswax. In another embodiment, a Gymnema-containing lip balm comprises cocoa butter. In another embodiment, a Gymnema-containing lip balm comprises shea butter. In another embodiment, a Gymnema-containing lip balm comprises tamanu oil. In another embodiment, a Gymnema-containing lip balm comprises avocado butter. In another embodiment, a Gymnema-containing lip balm comprises mango butter. In another embodiment, a Gymnema-containing lip balm comprises emu oil. In another embodiment, a Gymnema-containing lip balm comprises shea oil. In another embodiment, a Gymnema-containing lip balm comprises apricot kernel oil. In another embodiment, a Gymnema-containing lip balm comprises vitamin E. In another embodiment, a Gymnema-containing lip balm comprises almond oil. In another embodiment, a Gymnema-containing lip balm comprises castor oil. In another embodiment, a Gymnema-containing lip balm comprises lanolin. In another embodiment, a Gymnema-containing lip balm comprises sunflower oil. In another embodiment, a Gymnema-containing lip balm comprises a combination of the preceding substances. In another embodiment, a Gymnema-containing lip balm comprises ozokerite. In another embodiment, a Gymnema-containing lip balm comprises hydrogenated castor oil. In another embodiment, a Gymnema-containing lip balm comprises sorbitan oleate. In another embodiment, a Gymnema-containing lip balm comprises barley extract. In another embodiment, a Gymnema-containing lip balm comprises amur corktree bark extract. In another embodiment, a Gymnema-containing lip balm comprises sandalwood extract. In another embodiment, a Gymnema-containing lip balm comprises safflower seed oil. In another embodiment, a Gymnema-containing lip balm comprises aloe vera leaf extract. In another embodiment, a Gymnema-containing lip balm comprises sunflower seed oil. In another embodiment, a Gymnema-containing lip balm comprises rosemary leaf extract. In another embodiment, a Gymnema-containing lip balm comprises peppermint oil.

In one embodiment, a Gymnema-containing lip balm comprises Prunus amygdalus dulcis (sweet almond) oil, Cocos nucifera (coconut) oil, beeswax, Persea gratissima (avocado) oil, Theobroma cacao (cocoa) seed butter, and lanolin.

In another embodiment, a Gymnema-containing lip balm comprises Prunus amygdalus dulcis (sweet almond) oil, Cocos nucifera (coconut) oil, beeswax, Persea gratissima (avocado) oil, Theobroma cacao (cocoa) seed butter, and Butyrospermum parkii (shea butter).

In another embodiment, a Gymnema-containing lip balm comprises Prunus amygdalus dulcis (sweet almond) oil, Cocos nucifera (coconut) oil, beeswax, Persea gratissima (avocado) oil, Theobroma cacao (cocoa) seed butter, and Mangifera indica (mango) seed butter.

In another embodiment, a gynmema-containing lip balm comprises Ricinus communis (castor) seed oil, Elaeis guineensis (palm) kernel oil, Persea gratissima (avocado) oil, Cocos nucifera (coconut) oil, Aloe barbadensis leaf extract, Copernicia cerifera (carnauba) wax, and Euphorbia cerifera (candelilla) wax.

### EXAMPLES

Substances disclosed herein can be combined in specific formulations of Gymnema-containing lip balm or lip gloss. Although the following examples are hypothetical, the formulations are based on actual lip balm or lip gloss mix designs and are presented in this form in order to more completely illustrate the nature of the subject of this disclosure.

### Example 1

A Gymnema-containing lip balm can be formed from the following components:

| Ingredient | Mass (gram) |
|---|---|
| Gymnema Extract | X |
| Candelilla Wax | 9 |
| Carnauba Wax | 8 |
| Tamanu Oil | 5 |
| Avocado Butter | 10 |
| Mango Butter | 10 |
| Cocoa Butter | 14 |
| Emu Oil | 10 |
| Castor Oil | 10 |
| Shea Oil | 12 |
| Apricot Kernel Oil | 12 |
| Vitamin E Acetate | 1 |
| Peppermint Oil | 6 |

In the formulation of Example 1, the amount of Gymnema extract can be varied from between about 0.1 g to about 50 g to achieve a desired level of activity. Also, in the formulation of Example 1, essential oil or extract of jasmine, grapefruit, vanilla, lemon, oregano, ylang ylang, or a combination of these can be substituted for, or combined with, peppermint oil.

### Example 2

A Gymnema-containing lip balm can be formed from the following components:

| Ingredient | Mass (gram) |
|---|---|
| Gymnema Extract | X |
| Beeswax | 200 |
| Shea Butter | 260 |
| Cocoa Butter | 206 |
| Almond Oil | 38.5 |
| Vitamin E Acetate | 0.5 |
| Peppermint Oil | 0.15 |

In the formulation of Example 2, the amount of Gymnema extract can be varied from between about 7 g to about 212 g to achieve a desired level of activity. Also, in the formulation of Example 2, essential oil or extract of jasmine, grapefruit, vanilla, lemon, oregano, ylang ylang, or a combination of these can be substituted for, or combined with, peppermint oil.

### Example 3

A Gymnema-containing lip balm can be formed from the following components:

| Ingredient | Mass (gram) |
|---|---|
| Gymnema Extract | X |
| Beeswax | 6 |
| Shea Butter | 4.9 |
| Castor Oil | 6 |
| Almond Oil | 6 |
| Vitamin E Acetate | 0.25 |
| Peppermint Oil | 0.25 |

In the formulation of Example 3, the amount of Gymnema extract can be varied from between about 0.024 g to about 7.2 g to achieve a desired level of activity. In the formulation of Example 3, essential oil or extract of jasmine, grapefruit, vanilla, lemon, oregano, ylang ylang, or a combination of these can be substituted for, or combined with, peppermint oil.

### Example 4

A Gymnema-containing lip balm can be formed from the following components:

| Ingredient | Mass (gram) |
|---|---|
| Gymnema Extract | X |
| Carnauba Wax | 1 |
| Shea Butter | 4.9 |
| Castor Oil | 50 |
| Sunflower Oil | 50 |
| Peppermint Oil | 3 |

In the formulation of Example 4, the amount of Gymnema extract can be varied from between about 0.1 g to about 55 g to achieve a desired level of activity. Also, in the formulation of Example 4, essential oil or extract of jasmine, grapefruit, vanilla, lemon, oregano, ylang ylang, or a combination of these can be substituted for, or combined with, peppermint oil.

### Example 5

A Gymnema-containing lip balm can be formed from the following components:

| Ingredient | Mass (gram) |
|---|---|
| Gymnema Extract | X |
| Carnauba Wax | 10 |
| Mango Butter | 14 |
| Mango Oil | 14 |
| Coconut Oil | 28 |
| Macadamia Nut Oil | 28 |

In the formulation of Example 5, the amount of Gymnema extract can be varied from between about 0.1 g to about 50 g to achieve a desired level of activity.

### Example 6

A Gymnema-containing lip balm can be formed from the following components:

| Ingredient | Mass (gram) |
|---|---|
| Gymnema Extract | X |
| Beeswax | 25 |
| Shea Butter | 33 |
| Cocoa Butter | 33 |
| Almond Oil | 40 |
| Vitamin E Acetate | 1 |
| Peppermint Oil | 0.5 |

In the formulation of Example 6, the amount of Gymnema extract can be varied from between about 0.13 g to about 35 g to achieve a desired level of activity. In the formulation of Example 6, essential oil or extract of jasmine, grapefruit, vanilla, lemon, oregano, ylang ylang, or a combination of these can be substituted for, or combined with, peppermint oil.

### Example 7

A Gymnema-containing lip balm can be formed from the following components:

| Ingredient | Mass (gram) |
|---|---|
| Gymnema Extract | X |
| Beeswax | 33 |
| Olive Oil | 65 |
| Rosemary Oil | 2 |

In the formulation of Example 7, the amount of Gymnema extract can be varied from between about 0.1 g to about 50 g to achieve a desired level of activity.

### Example 8

A Gymnema-containing lip gloss can be formed from the following components:

| Ingredient | Mass (gram) |
|---|---|
| Gymnema Extract | X |
| Carnauba Wax | 1 |
| Castor Oil | 100 |
| Peppermint Oil | 1 |

In the formulation of Example 8, the amount of Gymnema extract can be varied from between about 0.1 g to about 50 g to achieve a desired level of activity. Also, in the formulation of Example 8, essential oil or extract of jasmine, grapefruit, vanilla, lemon, oregano, ylang ylang, or a combination of these can be substituted for, or combined with, peppermint oil.

### Example 9

A Gymnema-containing lip balm can be formed from the following components:

| Ingredient | Mass (gram) |
|---|---|
| Gymnema Extract | X |
| Beeswax | 1.6 |
| Aloe Butter | 1.6 |
| Avocado Butter | 1.6 |
| Mango Butter | 0.8 |
| Peppermint Oil | 0.5 |

In the formulation of Example 9, the amount of Gymnema extract can be varied from between about 0.0061 g to about 3.1 g to achieve a desired level of activity. Also, in the formulation of Example 9, essential oil or extract of jasmine, grapefruit, vanilla, lemon, oregano, ylang ylang, or a combination of these can be substituted for, or combined with, peppermint oil.

### Example 10

A Gymnema-containing lip balm can be formed from the following components:

| Ingredient | Mass (gram) |
|---|---|
| Gymnema Extract | X |
| Beeswax | 20 |
| Lanolin | 10 |
| Cocoa Butter | 15 |
| Sweet Almond Oil | 45 |
| Coconut Oil | 10 |
| Peppermint Oil | 1.5 |

### Example 11

A Gymnema-containing lip balm can be formed from the following components:

| Ingredient | Mass (gram) or Amount |
|---|---|
| Gymnema sylvestre leaf extract | About 4.5 Percent |
| Ricinus communis (castor) seed oil | Greater Than About 1 Percent |
| Hydrogenated castor oil | Greater Than About 1 Percent |
| Ozokerite | Greater Than About 1 Percent |
| Euphorbia cerifera (candelilla) wax | Greater Than About 1 Percent |
| Sorbitan oleate | Greater Than About 1 Percent |
| Butyrospermum parkii (shea butter) | Less Than About 1 Percent |
| Prunus amygdalus dulcis | Less Than About 1 Percent |
| (sweet almond) oil | |
| Hordeum distichon (barley) extract | Less Than About 1 Percent |
| Mentha piperita (peppermint) oil | Less Than About 1 Percent |
| Santalum album (sandalwood) | Less Than About 1 Percent |
| wood extract | |
| Phellodendron amurense (amur | Less Than About 1 Percent |
| corktree) bark extract | |
| Carthamux tinctorious (safflower) | Less Than About 1 Percent |
| seed oil | |
| Aloe barbadenis (aloe vera) leaf extract | Less Than About 1 Percent |
| Helianthus annus (sunflower) seed oil | Less Than About 1 Percent |
| Rosmarinus officinalis (rosemary) | Less Than About 1 Percent |
| leaf extract | |

In the formulation of Example 11, the amount of Gymnema extract can be varied from between about 0.1 g to about 50 g to achieve a desired level of activity. Specifically, a Gymnema powder comprises about 4.5% of the mass of the lip balm. In the formulation of Example 11, essential oil or extract of jasmine, grapefruit, vanilla, lemon, oregano, ylang ylang, or a combination of these can be substituted for, or combined with, peppermint oil. The formulation of Example 11 can be prepared such that the formulation results in an opaque product that has a dark green color, and an earthy and minty odor.

Generally, the preceding formulations may be produced by melting any room temperature solids, combining and mixing any room temperature oils, adding a Gymnema extract, and adding one or more essential oils or extract, one or more flavor oils or extracts, and any coloring agent. The function of the Gymnema extract, essential oils and flavor oils may be damaged if they are added to a hot mixture. As such, these components should be added when the mixture is as cool as is practical.

Any methods disclosed herein comprise one or more steps or actions for performing the described method. The method steps and/or actions may be interchanged with one another. In other words, unless a specific order of steps or actions is required for proper operation of the embodiment, the order and/or use of specific steps and/or actions may be modified.

Without further elaboration, it is believed that one skilled in the art can use the preceding description to utilize the present disclosure to its fullest extent. The examples and embodiments disclosed herein are to be construed as merely illustrative and not a limitation to the scope of the present disclosure in any way. It will be apparent to those having skill in the art that changes may be made to the details of the above-described embodiments without departing from the underlying principles of the disclosure described herein. In other words, various modifications and improvements of the embodiments specifically disclosed in the description above are within the scope of the appended claims.

1. A composition for application to a person's lips, comprising an extract of at least one member of the plant family Asclepiadaceae.

2. The composition of claim 1, wherein the extract of at least one member of the plant family Asclepiadaceae comprises at least one individual component of an extract of the plant family Asclepiadaceae.

3. The composition of claim 1, wherein the at least one extract comprises an extract of the plant Gymnema sylvestre.

4. The composition of claim 3, wherein the composition comprises between about 0.1% and about 50% by weight Gymnema sylvestre extract.

5. The composition of claim 2, wherein the at least one individual component of an extract of the plant family Asclepiadaceae comprises gymnemic acid.

6. The method of claim 5, wherein the composition comprises between about 0.1% and about 50% by weight gymnemic acid.

7. The composition of claim 1, wherein the composition comprises a lip balm.

8. The composition of claim 1, wherein the composition comprises a lip stick.

9. The composition of claim 1, further comprising at least one wax.

10. The composition of claim 9, wherein the wax comprises candelilla wax, ozokerite, or a combination of candelilla wax and ozokerite.

11. The composition of claim 9, further comprising at least one fat.

12. The composition of claim 11, wherein the fat comprises shea butter.

13. The composition of claim 11, further comprising at least one oil.

14. The composition of claim 13, wherein the oil comprises castor seed oil, hydrogenated castor oil, or a combination of both castor seed oil and hydrogenated castor oil.

15. The composition of claim 13, further comprising at least one emulsifier.

16. The composition of claim 15, wherein the emulsifier comprises sorbitan oleate.

17. The composition of claim 15, wherein the composition further comprises a substance selected from the group consisting of jasmine, grapefruit, vanilla, lemon, peppermint, oregano, ylang ylang, sweet almond oil, barley extract, sandalwood extract, amur corktree bark extract, aloe vera, sunflower seed oil, rosemary, and combinations of the preceding substances.

18. The composition of claim 17, wherein the wax comprises candelilla wax, ozokerite, or a combination of candelilla wax and ozokerite.

19. The composition of claim 17, wherein the fat comprises shea butter.

20. The composition of claim 17, wherein the oil comprises castor seed oil, hydrogenated castor oil, or a combination of both castor seed oil and hydrogenated castor oil.

21. The composition of claim 17, wherein the emulsifier comprises sorbitan oleate.

22. A composition comprising:
between about 0.1% and about 50% by weight Gymnema sylvestre extract;
greater than about 1 percent by weight oil;
greater than about 1 percent by weight wax;
greater than about 1 percent by weight emulsifier; and,
less than about 1 percent by weight of one or more essential oils.

23. The composition of claim 22, wherein each of the one or more essential oils comprises between about 0.0001% and about 0.9999%, by weight.

24. The composition of claim 22, wherein the composition comprises a substance selected from the group consisting of jasmine, grapefruit, vanilla, lemon, peppermint, oregano, ylang ylang, sweet almond oil, barley extract, sandalwood extract, amur corktree bark extract, aloe vera, sunflower seed oil, rosemary, and combinations of the preceding substances.

25. The composition of claim 24, wherein the composition comprises a substance selected from the group consisting of jasmine, grapefruit, vanilla, lemon, peppermint, oregano, ylang ylang, sweet almond oil, barley extract, sandalwood extract, amur corktree bark extract, aloe vera, sunflower seed oil, rosemary, and
wherein each of the preceding substances comprises no more than 0.9999% by weight of the composition.

26. A method for reducing a person's body weight, comprising the steps of:
providing a composition comprising an extract of at least one member of the plant family Asclepiadaceae, wherein the composition is configured to be applied to a person's lips; and,
applying the composition prior to consuming a food item containing sugar.

27. The method of claim 26, wherein the extract of at least one member of the plant family Asclepiadaceae comprises an extract of the plant Gymnema sylvestre.

28. The method of claim 27, wherein the composition comprises a lip balm.

29. The method of claim 27, wherein the composition comprises a lip stick.

30. The method of claim 27, wherein the composition comprises between about 0.1% and about 50% by weight Gymnema sylvestre extract.

31. The method of claim 27, wherein the composition comprises between about 0.1% and about 50% by weight gymnemic acid.

32. The method of claim 25, further comprising at least one wax.

33. The method of claim 32 wherein the wax comprises candelilla wax, ozokerite, or a combination of candelilla wax and ozokerite.

34. The method of claim 32, further comprising at least one fat.

35. The method of claim 34, wherein the fat comprises shea butter.

36. The method of claim 34, further comprising at least one oil.

37. The method of claim 36, wherein the oil comprises castor seed oil, hydrogenated castor oil, or a combination of both castor seed oil and hydrogenated castor oil.

38. The method of claim 36, further comprising at least one emulsifier.

39. The method of claim 38, wherein the emulsifier comprises sorbitan oleate.

40. The composition of claim 38, wherein the composition further comprises a substance selected from the group consisting of jasmine, grapefruit, vanilla, lemon, peppermint, oregano, ylang ylang, sweet almond oil, barley extract, sandalwood extract, amur corktree bark extract, aloe vera, sunflower seed oil, rosemary, and combinations of the preceding substances.

41. The composition of claim 40, wherein the wax comprises candelilla wax, ozokerite, or a combination of candelilla wax and ozokerite.

42. The composition of claim 40, wherein the fat comprises shea butter.

43. The composition of claim 40, wherein the oil comprises castor seed oil, hydrogenated castor oil, or a combination of both castor seed oil and hydrogenated castor oil.

44. The composition of claim 40, wherein the emulsifier comprises sorbitan oleate.

45. A method for preparing a composition comprising a Gymnema sylvestre extract, comprising the steps of:
providing a Gymnema sylvestre extract;
providing a room temperature solid;
combining the Gymnema sylvestre extract with the room temperature solid.

46. The method of claim 45, wherein the room temperature solid comprises a wax.

47. The method of claim 45, wherein the room temperature solid comprises a butter.

48. The method of claim 45, wherein combining the Gymnema sylvestre extract with the room temperature solid comprises melting the room temperature solid, adding the extract, and mixing the two.

49. The method of claim 45, further comprising the steps of:
providing an extract selected from the group consisting of jasmine, grapefruit, vanilla, lemon, peppermint, oregano, ylang ylang, and combinations of the preceding substances; and,
combining the substance with the Gymnema sylvestre extract and the room temperature solid.

50. The method of claim 45, further comprising the steps of:
providing at least one oil; and,
combining the at least one oil with the Gymnema sylvestre extract and the room temperature solid.
